(19) United States
(12) Patent Application Publication
(10) Pub. No.: US 2010/0226818 A1
(43) Pub. Date: Sep. 9, 2010

(54) MULTIPLE-FUNCTION DEODORANT DISPENSER APPARATUS AND METHODS

(75) Inventors: Haruyoshi Miyagi, Lehi, UT (US); Daniel J. Rodrique, Herriman, UT (US); David Bernstein, Park City, UT (US); Christian Weaver, Eagle Mountain, UT (US)

Correspondence Address: ALAN J. HOWARTH, P.O. BOX 1909, SANDY, UT, 84091-1909, US

(73) Assignee: F-MATIC, INC., Lehi, UT (US)

(21) Appl. No.: 12/716,960

(22) Filed: Mar. 3, 2010

Related U.S. Application Data
(60) Provisional application No. 61/157,094, filed on Mar. 3, 2009.

(51) Int. Cl. A61L 9/00; B65D 83/44; A61L 9/14; A61L 9/015

(52) U.S. Cl. 422/4; 222/402.1; 422/124

(57) ABSTRACT

A multiple-purpose deodorant dispenser unit is provided, comprising: (a) first apparatus adapted for alternatively receiving (i) an aerosol spray deodorant container, (ii) an open deodorant container having gel, liquid or solid deodorant therein, or (iii) a wick cylinder having deodorant therein; (b) second apparatus adjacent to the first apparatus adapted for alternatively containing (i) a spray actuating head device for activating a nozzle in the aerosol spray deodorant container to emit a spray of vaporized deodorant, or (ii) a fan head device for directing a movement of air in the vicinity of either the open container or the wick cylinder; and (c) a third apparatus having electrical means for alternatively operating the spray actuating head device or the fan head device.

A method for dispensing deodorant using a multiple-purpose deodorant dispenser unit is provided, comprising (a) alternatively placing in the multiple-purpose dispenser unit (i) an aerosol spray deodorant container, (ii) an open deodorant container having gel, liquid or solid deodorant therein, or (iii) a wick cylinder having deodorant therein; (b) alternatively placing in the multiple-purpose dispenser unit (i) a spray actuating head device for activating a nozzle in the aerosol spray deodorant container to emit a spray of vaporized deodorant, or (ii) a fan head device for directing a movement of air in the vicinity of either the open deodorant container or the wick cylinder; and (c) placing an electrical means in the multiple-purpose dispenser unit for alternatively causing the electrical means to operate the spray actuating head device or to operate the fan head device.

### CROSS REFERENCE TO RELATED APPLICATIONS

This application claims the benefit of U.S. Provisional Application No. 61/157,094, filed Mar. 3, 2009, which is hereby incorporated by reference.

### STATEMENT REGARDING FEDERALLY SPONSORED RESEARCH

Not applicable.

### BACKGROUND OF THE INVENTION

In public restrooms, such as in commercial buildings and restaurants, deodorant apparatus is used to remove or mask unpleasant odors. This deodorant apparatus comes in many types and sizes. One common device involves the use of a dispenser that activates an aerosol spray dispenser to spray various types of fragrances. Another device uses a fan to direct air into a restroom that is scented by a deodorant gel or wick. Some units operate using batteries, others connect to an electrical outlet or rely on movement of air in a room to dissipate deodorant.

It is not unusual for restroom maintenance personnel to decide to change from one type or brand to another one. In such situations, different types or brands of dispensers are usually not compatible with freshener dispensers or products previously used. In such a case, when a different type of dispenser is installed, the previously used unit is often not removed. Consequently, it is common to see several different dispensers cluttering a restroom wall, in various stages of disrepair or nonuse.

In addition, changing to different types or brands of dispensers can be time consuming. Further, if a purchaser decides to change back to a previously used dispenser, it is likely that the old dispenser requires repair, cleaning or updating before it can be used again.

### SUMMARY OF THE INVENTION

A multiple-purpose deodorant dispenser unit is provided, comprising: (a) first apparatus adapted for alternatively containing (i) an aerosol spray deodorant container, (ii) an open deodorant container having gel, liquid or solid deodorant therein, or (iii) a wick cylinder having deodorant therein; (b) second apparatus adjacent to the first apparatus adapted for alternatively containing (i) a spray actuating head device for activating a nozzle in the aerosol spray deodorant container to emit a spray of vaporized deodorant, or (ii) a fan head device for directing a movement of air in the vicinity of either the open container or the wick cylinder; and (c) a third apparatus having electrical means for alternatively operating the spray actuating head device or the fan head device.

A method for dispensing deodorant using a multiple-purpose deodorant dispenser unit is provided, comprising (a) alternatively placing in the multiple-purpose dispenser unit (i) an aerosol spray deodorant container, (ii) an open deodorant container having gel, liquid or solid deodorant therein, or (iii) a wick cylinder having deodorant therein; (b) alternatively placing in the multiple-purpose dispenser unit (i) a spray actuating head device for activating a nozzle in the aerosol spray deodorant container to emit a spray of vaporized deodorant, or (ii) a fan head device for directing a movement of air in the vicinity of either the open deodorant container or the wick cylinder; and (c) placing an electrical means in the multiple-purpose dispenser unit for alternatively causing the electrical means to operate the spray actuating head device or to operate the fan head device.

### BRIEF DESCRIPTION OF DRAWINGS

In the detailed description that follows, reference will be made to the following Figures, in which:

FIGS. 1A and 1B are perspective views of a multiple-purpose deodorant dispensing unit according to the described embodiments of the present invention;

FIG. 2 is a perspective, partially fragmented, view of a rear housing of the deodorant dispensing unit shown in FIGS. 1A and 1B, having an aerosol spray deodorant container mounted therein;

FIG. 3 is a perspective view of the rear housing of the deodorant dispensing unit shown in FIG. 2;

FIG. 4 is a perspective view of a spray actuating head mounted in the rear housing of the deodorant dispensing unit shown in FIG. 2;

FIG. 5 is a perspective view of a rear housing of the deodorant dispensing unit shown in FIGS. 1A and 1B, having a fan head and deodorant container mounted therein;

FIG. 6 is a perspective view of the fan head mounted in the rear housing of the deodorant dispensing unit shown in FIG. 5;

FIGS. 7 and 8 are perspective views of the fan head shown in FIGS. 5 and 6; and

FIG. 9 is a perspective, partially fragmented, view of a rear housing of the deodorant dispensing unit shown in FIGS. 1A and 1B, having a fan head and deodorant wick cylinder mounted therein.

### DETAILED DESCRIPTION OF THE DISCLOSURE

The present invention, as described in the following embodiments, provides a number of advantages over the existing prior art. The multiple-purpose deodorant dispensing unit disclosed herein is light and easy to manufacture, ship and install.

Moreover, once installed, the multiple-purpose dispensing unit may be easily changed to use a spray actuating head for activating an aerosol spray container, or to use a battery-operated fan for directing deodorants into a room from an open deodorant container. Alternatively, a deodorant wick cylinder may be provided to be used with the battery-operated fan or to operate without the battery and fan by simply using existing air currents to direct deodorants into the restroom, such as air currents generated by the restroom door being opened and closed.

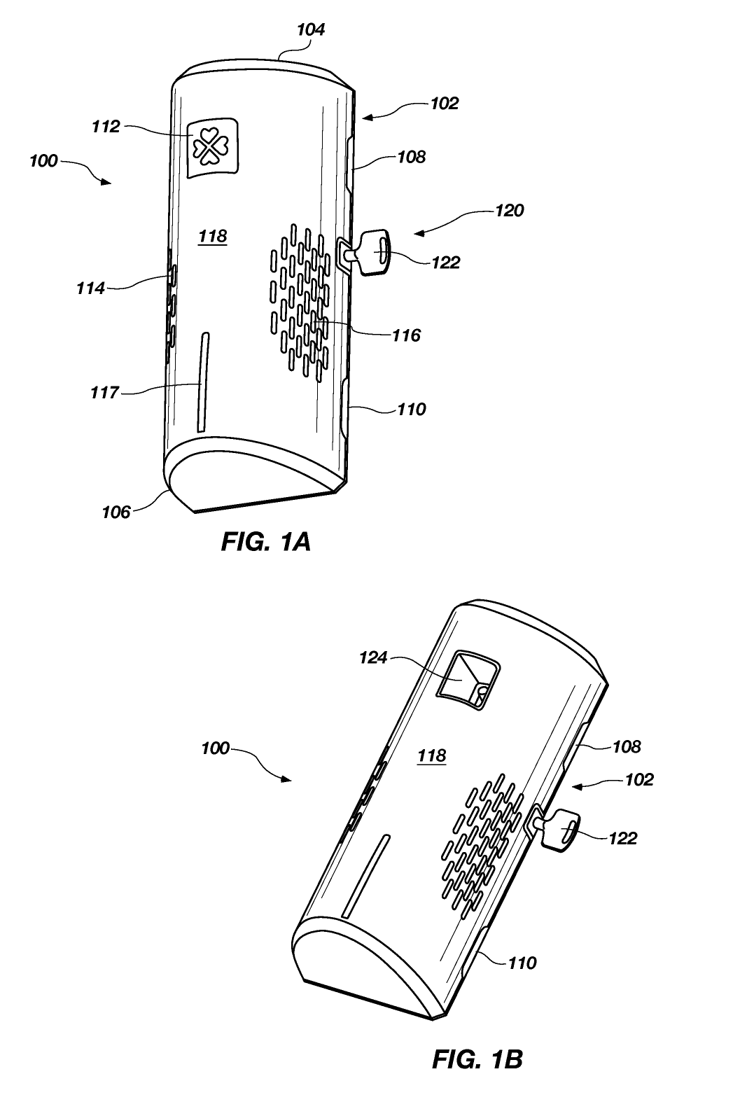

Referring to FIGS. 1A and 1B, an embodiment of the present invention is shown in the form of a multiple-function deodorant dispenser unit 100. Unit 100 comprises a tubular-section shaped front section 102 and a flat rear housing (not shown here). Unit 100 further comprises flat top and 15 bottom pieces 104 and 106, respectively. Slots 108 and 110 run along the base of tubular-shaped front section 102, providing access for air to flow out of dispenser 100. Corresponding slots (not shown) are provided on a side opposite slots 106 and 110.

A decorator tab 112 on the face 118 of unit 100 closes off an opening (not shown here) for aerosol spray. A lock and key unit 120 having a key 122 locks the front section 102 to the rear housing. Decorator indentations 114 and 116 and a decorator strip 117 are formed on the sides and front of face 118 of the front section 102. FIG. 1B shows the multiple-function deodorant dispenser unit 100 with the decorator tab 112 removed to expose an aperture 124 in the face 118 of front section 102 for directing aerosol spray.

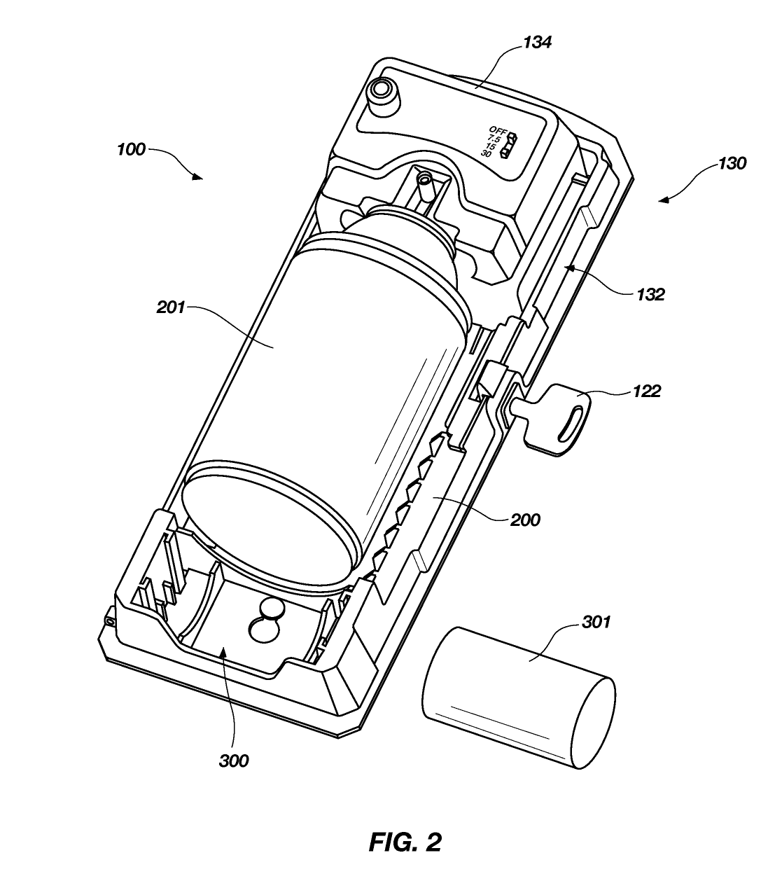

Referring now to FIG. 2, multiple-function deodorant dispenser unit 100 is shown with the front section 102 removed by turning key 122. Rear housing 130 includes an upper bay 132 for receiving a spray actuator head 134 and a center bay 200 for receiving an aerosol spray container 201. Housing section 130 also includes a lower bay 300 for receiving a battery 301.

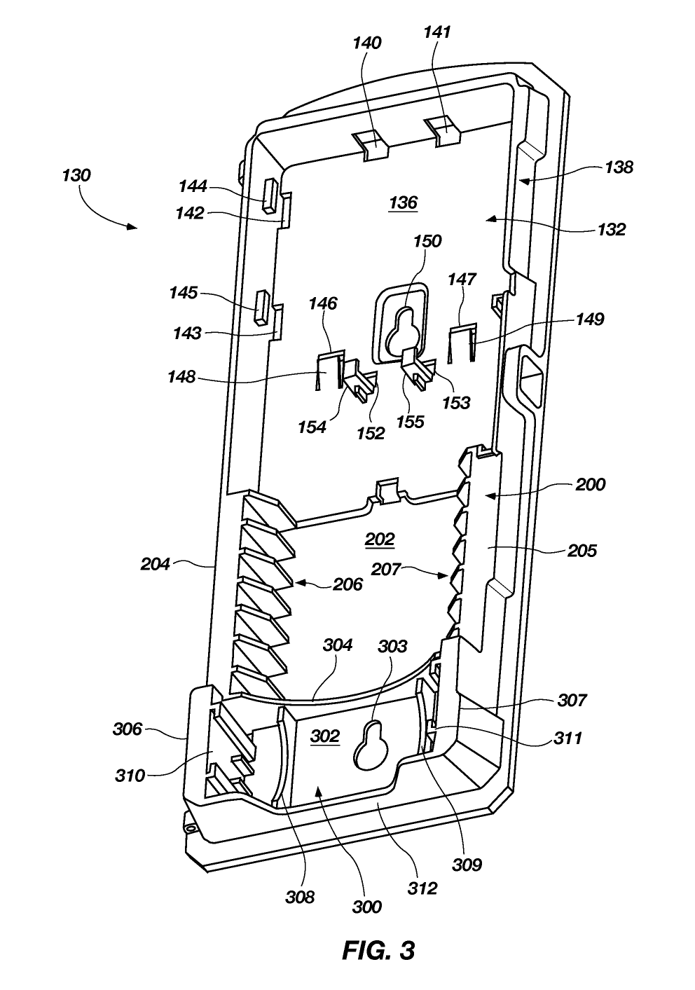

Looking next at FIG. 3, rear housing 130 is shown to comprise a flat bottom 136 and a shallow wall 138 running around three sides of 136. Upper bay 132 includes small rectangular upper openings 140 and 141 notched between bottom 136 and wall 138. Small rectangular side openings 142 and 143 are formed on a portion of bottom 136 adjoining wall 138. Similar side openings (not shown) are formed directly opposite of side openings 142 and 143 where bottom 136 adjoins wall 138. Two side nubs 144 and 145 are formed on an inside portion of wall 138 directly above side openings 142 and 143. Two additional corresponding nubs (not shown) are also formed on an inside portion of wall 138 opposition of tabs 144 and 145.

In addition, rectangular lower openings 146 and 147 are formed near the bottom of bay 132, with corresponding flexible fingers 148 and 149 extending over openings 146 and 147. The openings 146, 147, tabs 144, 145 and flexible fingers 148, 149 described above are useful for securing spray actuating head 134 in a manner to be described below. An opening 150 is provided in bottom 136 for a screw to attach rear housing 130 to a vertical wall or other surface (not shown). Openings 152 and 153 are also shown near the bottom of bay 132, to accommodate wiring from the battery 301, in a manner to be described later. Hooks 154 and 155 extend upward above openings 152 and 153 for attaching wiring to make contact with electrical contacts (not shown) on spray actuating head 134.

FIG. 3 also shows center bay 200 in more detail, including a bottom 202 that is a continuation of bottom 136 in upper bay 130. Central bay 200 also includes sidewalls 204 and 205, each having a series of parallel pairs of notches or ledges 206 and 207 therein.

FIG. 3 also shows the lower bay 300 in more detail, including a bottom 302 separated from bottom 202 by a curved wall 304. Sidewalls 306 and 307 and end wall 312 further comprise the formation of lower bay 300. Within bay 300 opposing cradle walls 308 and 309 are vertically formed rising up from bottom 302 to hold battery 301 (not shown here). On the inside of walls 306 and 307 are slots 310 and 311 for holding contacts (not shown) to connect with the battery (not shown here). An opening 303 is provided for a screw that connects rear housing to a wall or other surface.

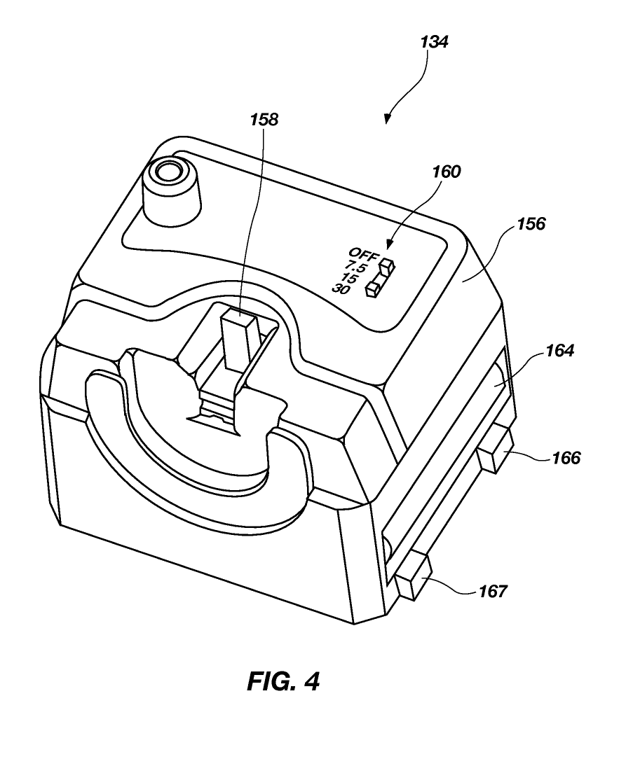

FIG. 4 shows the spray actuating head 134 in greater detail. An upper head portion 156 includes conventional internal timing circuitry therein (not shown) for operating a spray nozzle 158 that is usually affixed to an aerosol spray container (not shown here). The internal timing circuitry includes a conventional timing device (not shown) such as a gear that will rotate and actuate nozzle 158 by forcing it downward briefly towards the aerosol spray container to cause a short emission of vaporized deodorant from nozzle 158. A switch 160 enables selection of different intervals (such as 7.5 minutes, 15 minutes or 30 minutes) between times when the nozzle 158 is actuated.

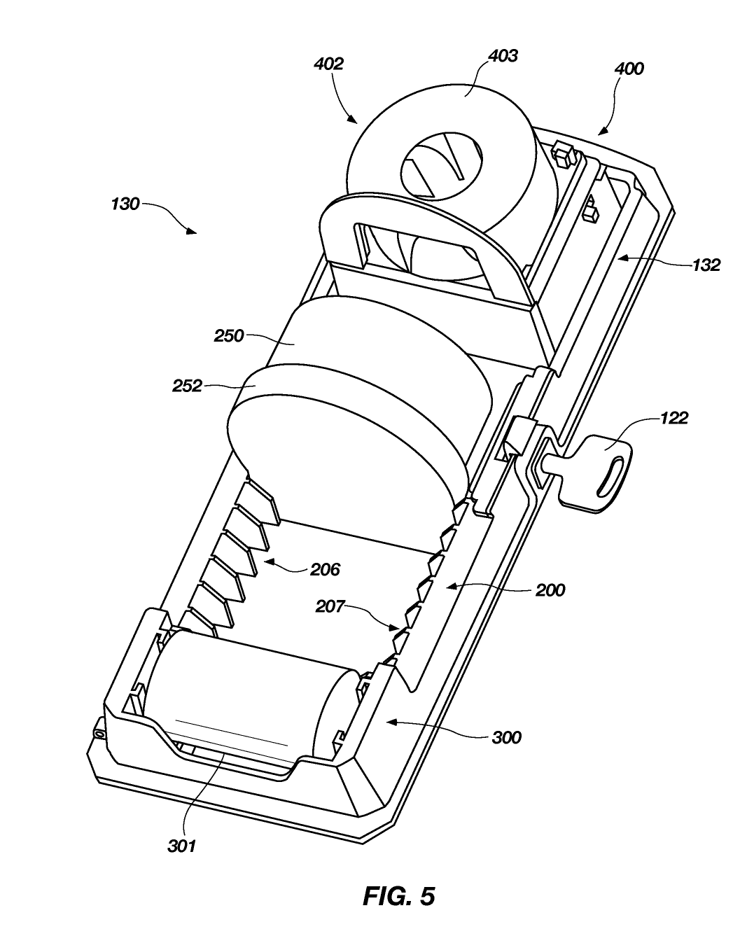

A base section 164 of spray actuating head 134 includes tabs 166 and 167 that are made to engage with corresponding openings (not shown) in the bottom 136 of rear housing 130. In addition, other similar tabs (not shown) are formed in other portions of spray actuating head 134, to attach into the upper openings 140 and 141 and side openings 142 and 143 shown in FIG. 3, so as to secure spray actuating head 134 to rear housing 130. FIG. 5 shows an alternate embodiment of the present invention, in which the rear housing 130 functions as a fan-operated dispenser of deodorant from an open container. In this embodiment, a fan head 400 having a circular fan 402 is disposed in the upper bay 132 instead of a spray actuating head. The openings 146, 147, tabs 144, 145 and flexible fingers 148, 149 described with respect to FIG. 3 above in connection with securing spray actuating head 134, are also used to secure fan head 402 in the same manner. Battery 301 is disposed in lower bay 300 to operate the fan 402 by means of electrical wiring (not shown).

An open deodorant container 250 of solid, liquid or gel deodorant is disposed in center bay 200 rather than an aerosol spray container. A circular shelf 252 is disposed in a pair of the sets of parallel ledges 206 and 207 to support container 250. As shown in FIG. 3, multiple pairs of parallel ledges 206, 207 are provided to assist in regulating the strength of the fragrance being emitted from open deodorant container 250 positioned on shelf 252. The amount of fragrance being directed from open deodorant container 250 by fan 402 may be increased by positioning shelf 252 on a pair of parallel ledges 206, 207 closer to the fan 402. Conversely, the amount of fragrance being directed from deodorant container 250 by fan 402 may be decreased by positioning shelf 252 on a pair of parallel ledges 206, 207 further from fan 402. Alternatively, a single support piece (not shown) may be used to support shelf 252 at one position relative to fan head 402.

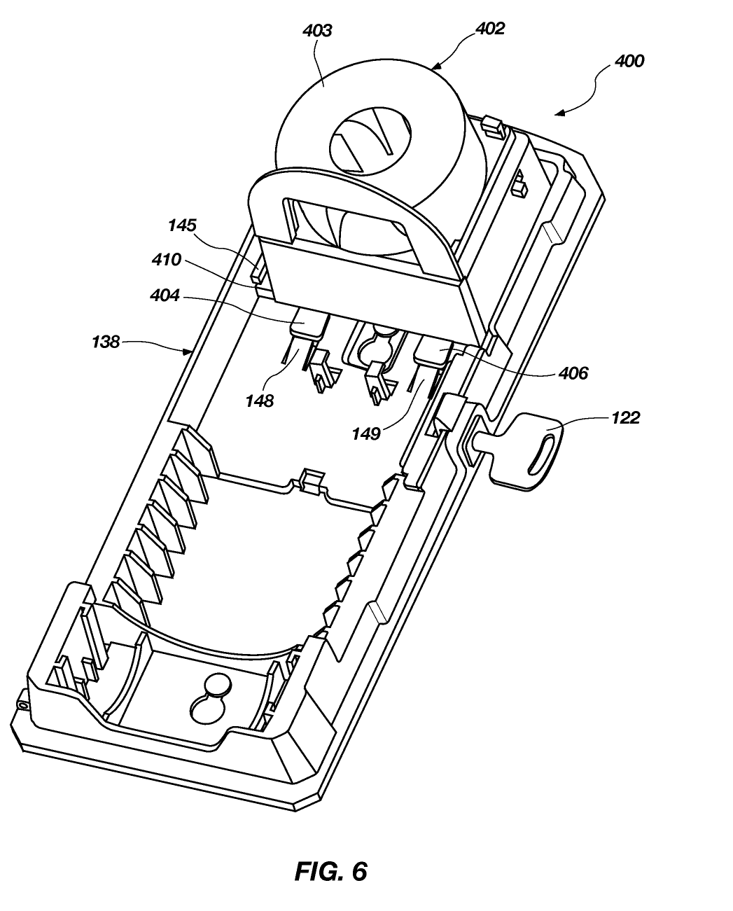

FIG. 6 shows the rear housing 130 without the deodorant container 250 and shelf 252, which are removed from central bay 200. Fan head 400 is attached into upper bay 132 by lower tabs 404 and 406 abutting flexible fingers 148 and 149. Fan head 400 also has side tabs on both sides of fan head 400 that are secured by corresponding side nubs on wall 138 of rear housing 130. Only side nub 145 is shown, which abuts side tab 410 of fan head 400.

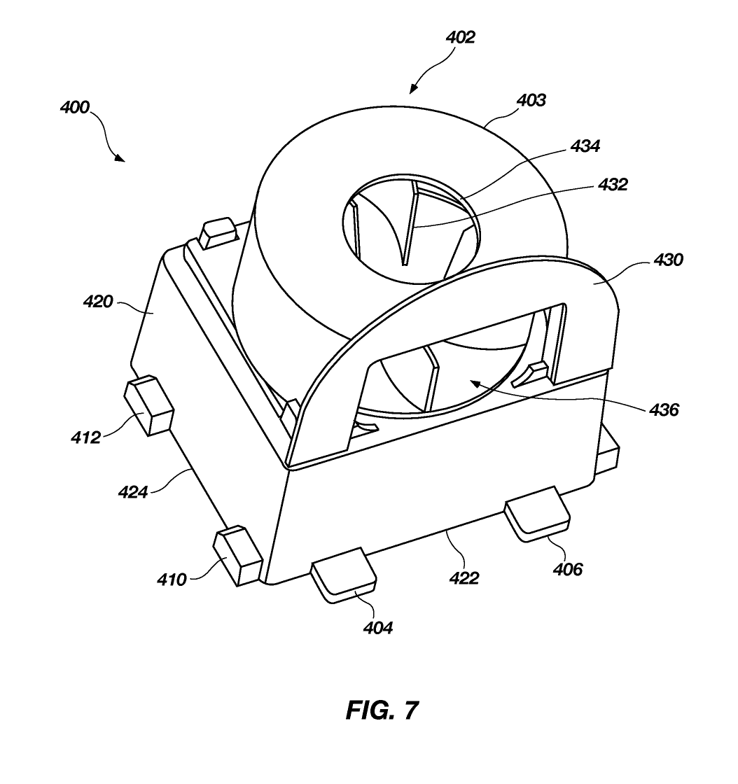

Looking now at FIG. 7, the fan head 400 is shown in greater detail. A round fan 402 includes a fan housing 403 disposed on a base 420. Lower tabs 404 and 406 extend from a lower portion 422 of fan head 400. Side tabs 410 and 412 extend from a side portion 424 of fan head 400. Corresponding pairs of tabs extend from the other two walls (not shown here) of fan head 400. A handle 430 extends upward from base 420. Within the fan head 402 are curved blades 432. An air hole 434 is disposed in the top of fan 402. As the blades 432 rotate, air is pulled through air hole 434 and dispersed through a large opening 436 in the fan housing 403.

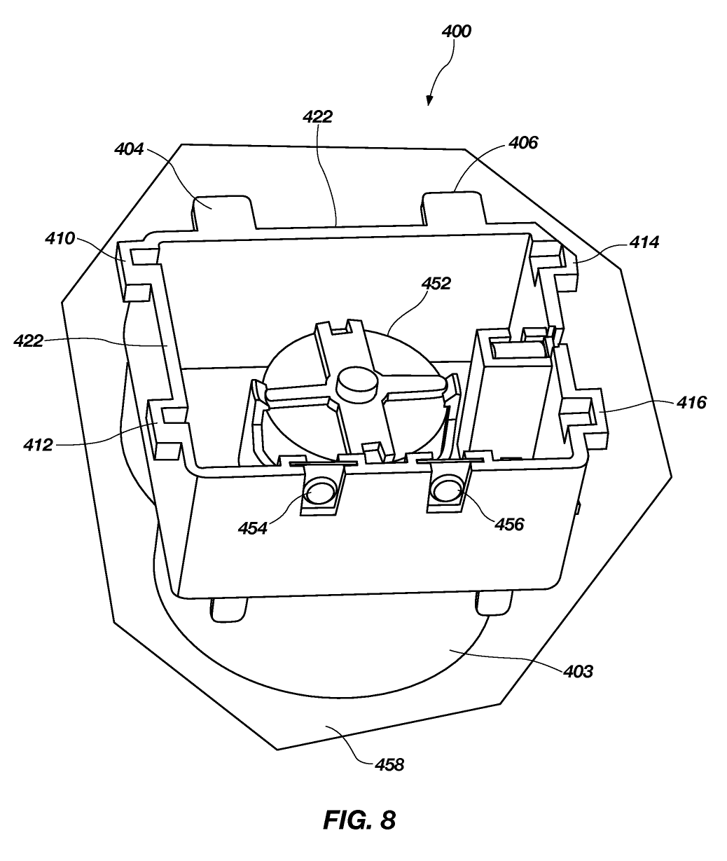

FIG. 8 shows an inverted view of fan head 400 disclosing the underside 450 of fan head 400. A circular base 452 of the fan blades 432 is shown, as well as lower tabs 404 and 406 and side tabs 410 and 412. In addition, corresponding side tabs 414 and 416 are shown, which also serve to secure the fan head 400 in upper bay 130. A pair of circular electrical touch pads 454 and 456 are located on a lower portion of upper side 458 of fan head 400 to connect with corresponding electrical outlets (not shown) in wall 138 of upper bay 132, that electrically communicate by wires running to battery 301. Alternatively, electrical touch pads 454 and 456 may comprise metal springs, wire connectors or other means to connect electrically to wires running to battery 301.

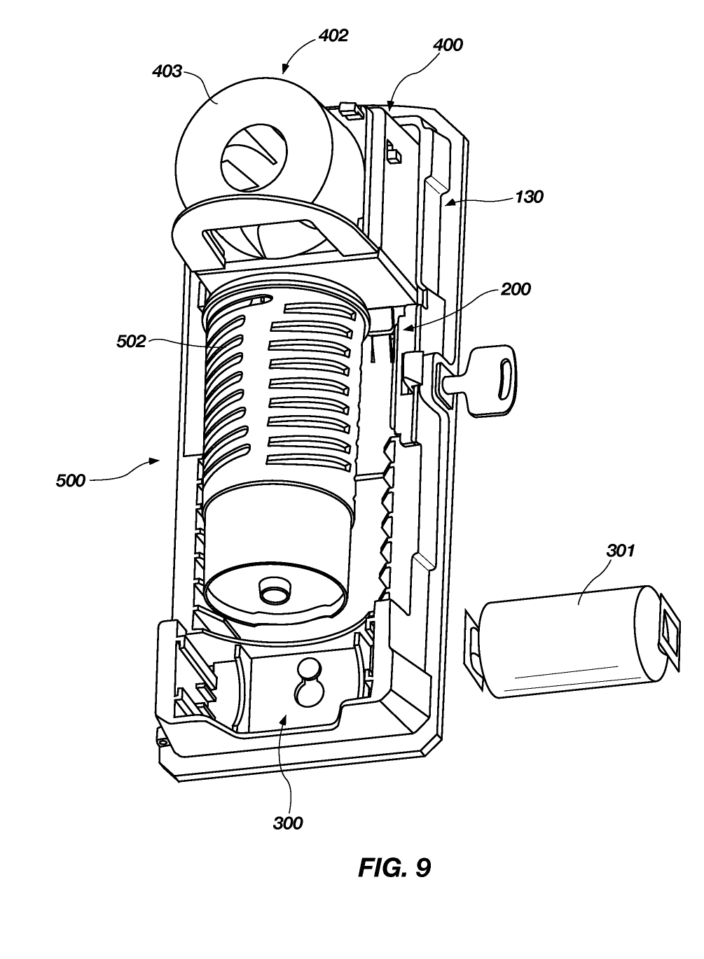

FIG. 9 shows another function of the present invention in which the rear housing 130 functions as a fan-operated dispenser of deodorant from a wick container. In this embodiment, fan head 400 is disposed in upper bay 132. A conventional liquid deodorant wick container 500 is provided in center bay 200 having liquid deodorant and a wick therein for conveying the liquid deodorant to external vents 502. Battery 301 is disposed in lower bay 300 to operate the fan 402 of fan head 400. As the fan 402 rotates, deodorant is conveyed from vents 502 of wick container 500 through external air vents 108 and 110 (see FIGS. 1A and 1B). Alternatively, battery 301 and fan head 400 may be removed, so that the normal movement of air in a restroom, for example by the opening and closing of doors therein, will cause sufficient air currents to convey deodorant from the wick container 500 through external air vents 108 and 110 to the room.

The present invention, as described in the foregoing embodiments, provides a number of advantages over the existing prior art. The multiple-purpose deodorant dispensing unit disclosed herein is light and easy to manufacture, ship and install. Moreover, once installed, the multiple-purpose dispensing unit may be easily changed to use a spray actuating head for activating an aerosol spray container or, as an alternative, to use a battery-operated fan for directing deodorants into a room from a deodorant container. Another alternative is provided in which a deodorant wick cylinder is used with the battery-operated fan to direct deodorant into a restroom. A variation of this alternative is to operate the wick unit without a battery and fan by simply using existing air currents to direct deodorants into the restroom. Thus, one multiple function unit may be used to alternatively function as multiple different types of deodorant systems for refreshing air in a room.

The spray actuating head 134 and fan head 400 may be conventional units, either made for the 15 current embodiments or bought from external sources. Likewise the aerosol spray containers of compressed deodorants and the containers of liquid, solid or gel deodorants are conventional units available on the market. The wick cylinder shown herein is also conventional and may be purchased from a variety of sources. The above embodiments may be connected to an electrical outlet through a portable transformer rather than using a portable battery as shown.

Various changes in materials and components can be made within the spirit and coverage of the present invention. Most components shown herein may be made of plastic, metal or other suitable materials. The fragrances may be made of any suitable chemicals for use as described herein.

It will be understood that the invention may be embodied in other specific forms by one of ordinary skill in the art without departing from the spirit, characteristics or coverage of the present invention. The present example and embodiment are to be considered to be illustrative and not restrictive, and the invention is not intended to be limited to the details of the described embodiments. Rather, the invention is defined by the claims, and as broadly as the prior art will permit.

What is claimed is:

1. A multiple-purpose deodorant dispenser unit, comprising:
(a) first apparatus adapted for alternatively receiving (i) an aerosol spray deodorant container, (ii) an open deodorant container having gel, liquid or solid deodorant therein, or (iii) a wick cylinder having deodorant therein;
(b) second apparatus adjacent to the first apparatus adapted for alternatively receiving (i) a spray actuating head device for activating a nozzle in the aerosol spray deodorant container to emit a spray of vaporized deodorant, or (ii) a fan head device for directing a movement of air in the vicinity of either the open deodorant container or the wick cylinder; and
(c) a third apparatus having electrical means for alternatively operating the spray actuating head device or the fan head device.

2. The multiple-purpose deodorant dispensing unit of claim 1, wherein the first apparatus comprises a first bay in a housing unit, the second apparatus comprises a second bay adjacent to the first bay in the housing unit, and the third apparatus comprises a third bay in the housing unit.

3. The multiple-purpose deodorant dispensing unit of claim 1, wherein a spray actuating head device is disposed adjacent to the nozzle of the aerosol spray deodorant container to periodically depress the nozzle, thereby causing the nozzle to emit a spray of vaporized deodorant.

4. The multiple-purpose deodorant dispensing unit of claim 1, wherein the fan head is a circular fan having an opening therein adjacent to the open deodorant container or the wick cylinder for directing a current of air in the vicinity of the open container or the wick cylinder.

5. The multiple-purpose deodorant dispensing unit of claim 2, further comprising first support structure in the first bay for supporting the open deodorant container.

6. The multiple-purpose deodorant dispensing unit of claim 2, further comprising second support structure in the second bay for alternatively supporting the spray actuating head or the fan head in the second bay.

7. The multiple-purpose deodorant dispensing unit of claim 2, in which the first support structure has alternative positioning structure for supporting the open deodorant container at alternative positions relative to the fan head device.

8. A method for dispensing deodorant using a multiple-purpose deodorant dispenser unit, comprising:
(a) alternatively placing in the multiple-purpose dispenser unit (i) an aerosol spray deodorant container, (ii) an open deodorant container having gel, liquid or solid deodorant therein, or (iii) a wick cylinder having deodorant therein;
(b) alternatively placing in the multiple-purpose dispenser unit (i) a spray actuating head device for activating a nozzle in the aerosol spray deodorant container to emit a spray of vaporized deodorant, or (ii) a fan head device for directing a movement of air in the vicinity of either the open deodorant container or the wick cylinder; and
(c) alternatively connecting an electrical means in the multiple-purpose dispenser unit to the spray actuating head device or to the fan head device for alternatively causing the electrical means to operate the spray actuating head device or to operate the fan head device.

9. The method of claim 8, further comprising disposing the spray actuating head device adjacent to the nozzle of the aerosol spray deodorant container to periodically depress the nozzle, thereby causing the nozzle to emit a spray of vaporized deodorant.

10. The method of claim 8, wherein the fan head device comprises a fan having an opening therein, further comprising placing the fan head device with the opening adjacent to the open deodorant container or the wick cylinder for directing a current of air in the vicinity of the open deodorant container or the wick cylinder.

11. The method of claim 8, and further comprising alternatively positioning the open deodorant container at selected distances relative to the fan head device to regulate the amount of deodorant being emitted by the deodorant dispenser unit.

12. A multiple-purpose deodorant dispenser unit, comprising a housing having:
(a) a first bay for alternatively receiving (i) an aerosol spray deodorant container, (ii) an open deodorant container having gel, liquid or solid deodorant therein, or (iii) a wick cylinder having deodorant therein;
(b) a second bay adjacent to the first bay for alternatively receiving (i) a spray actuating head device for activating a nozzle in the aerosol spray deodorant container to emit a spray of vaporized deodorant, or (ii) a fan head device for directing a movement of air in the vicinity of either the open deodorant container or the wick cylinder; and
(c) a third bay having electrical means for alternatively operating the spray actuating head device or the fan head device.

13. The multiple-purpose deodorant dispenser unit of claim 12, further comprising a cover having connecting means to connect the cover to the housing and an opening for emitting the vaporized deodorant or the movement of air from the dispenser unit.

14. The multiple-purpose deodorant dispenser unit of claim 12, wherein the electrical means comprises a portable battery and electrical conduits for alternatively connecting the battery to the spray actuating head or to the fan head.

15. The multiple-purpose deodorant dispenser unit of claim 12, further comprising a ledge in the first bay for supporting the open deodorant container having gel, liquid or solid deodorant therein.

16. The multiple-purpose deodorant dispenser unit of claim 12, further comprising multiple pairs of ledges in the first bay for supporting the open deodorant container at different distances from the fan head device to regulate the amount of deodorant directed from the dispenser unit.
